(19) United States
(12) Patent Application Publication
(10) Pub. No.: US 2011/0218387 A1
(43) Pub. Date: Sep. 8, 2011

(54) ANCHORS FOR USE IN MEDICAL APPLICATIONS

(75) Inventors: Theodore C. Lamson, Pleasanton, CA (US); Joseph Catanese, III, San Leandro, CA (US); Jacqueline N. Welch, Moraga, CA (US); Michael Wei, Redwood City, CA (US)

(73) Assignee: NEOTRACT, INC., Pleasanton, CA (US)

(21) Appl. No.: 13/034,016

(22) Filed: Feb. 24, 2011

Related U.S. Application Data
(60) Provisional application No. 61/311,205, filed on Mar. 5, 2010.

(51) Int. Cl. A61F 2/04

(52) U.S. Cl. 600/30

(57) ABSTRACT

An anchor system and associated method for manipulating, approximating or compressing tissues and anatomical or other structures in medical applications for the purpose of treating diseases or disorders or other purposes. The anchor includes one or more elastic wing and fin structures extending radially from the anchor's body.

### BACKGROUND

The disclosed embodiments relate generally to medical devices and methods, and more particularly to systems and associated methods for manipulating or retracting tissues and anatomical or other structures within the body of human or animal subjects for the purpose of treating diseases or disorders.

There are a wide variety of situations in which it is desirable to lift, compress or otherwise reposition normal or aberrant tissues or anatomical structures (e.g., organs, ligaments, tendons, muscles, tumors, cysts, fat pads, and the like) within the body of a human or animal subject. Such procedures are often carried out for the purpose of treating or palliating the effects of diseases or disorders (e.g., hyperplasic conditions, hypertrophic conditions, neoplasias, prolapses, herniations, stenoses, constrictions, compressions, transpositions, congenital malformations, and the like) and/or for cosmetic purposes (e.g., face lifts, breast lifts, brow lifts, and the like) and/or for research and development purposes (e.g., to create animal models that mimic various pathological conditions). In many of these procedures, surgical incisions are made in the body, and laborious surgical dissection is performed to access and expose the affected tissues or anatomical structures. Thereafter, in some cases, the affected tissues or anatomical structures are removed or excised. In other cases, various natural or man-made materials are used to lift, sling, reposition or compress the affected tissues.

Benign Prostatic Hyperplasia (BPH)

One example of a condition where it is desirable to lift, compress or otherwise remove a pathologically enlarged tissue is Benign Prostatic Hyperplasia (BPH). BPH is one of the most common medical conditions that affect men, especially elderly men. It has been reported that, in the United States, more than half of all men have histopathologic evidence of BPH by age 60 and, by age 85, approximately 9 out of 10 men suffer from the condition. Moreover, the incidence and prevalence of BPH are expected to increase as the average age of the population in developed countries increases.

The prostate gland enlarges throughout a man's life. In some men, the prostatic capsule around the prostate gland may prevent the prostate gland from enlarging further. This causes the inner region of the prostate gland to squeeze the urethra. This pressure on the urethra increases resistance to urine flow through the region of the urethra enclosed by the prostate. Thus, the urinary bladder has to exert more pressure to force urine through the increased resistance of the urethra. Chronic over-exertion causes the muscular walls of the urinary bladder to remodel and become stiffer. This combination of increased urethral resistance to urine flow and stiffness and hypertrophy of urinary bladder walls leads to a variety of lower urinary tract symptoms (LUTS) that may severely reduce the patient's quality of life. These symptoms include weak or intermittent urine flow while urinating, straining when urinating, hesitation before urine flow starts, feeling that the bladder has not emptied completely even after urination, dribbling at the end of urination or leakage afterward, increased frequency of urination particularly at night, urgent need to urinate, and the like.

In addition to patients with BPH, LUTS may also be present in patients with prostate cancer, prostate infections, and chronic use of certain medications (e.g. ephedrine, pseudoephedrine, phenylpropanolamine, antihistamines such as diphenhydramine, chlorpheniramine, and the like) that cause urinary retention especially in men with prostate enlargement.

Although BPH is rarely life threatening, it can lead to numerous clinical conditions including urinary retention, renal insufficiency, recurrent urinary tract infection, incontinence, hematuria, and bladder stones.

Medications for treating BPH symptoms include phytotherapy and prescription medications. Surgical procedures for treating BPH symptoms include Transurethal Resection of Prostate (TURP), Transurethral Electrovaporization of Prostate (TVP), Transurethral Incision of the Prostate (TUIP), Laser Prostatectomy and Open Prostatectomy. Minimally invasive procedures for treating BPH symptoms include Transurethral Microwave Thermotherapy (TUMT), Transurethral Needle Ablation (TUNA), Interstitial Laser Coagulation (ILC), and Prostatic Stents.

Although existing treatments provide some relief to the patient from symptoms of BPH, they have disadvantages. Alpha-1 a-adrenergic receptors blockers have side effects such as dizziness, postural hypotension, lightheadedness, asthenia and nasal stuffiness. Retrograde ejaculation can also occur. 5-alpha-reductase inhibitors have some side effects, such as weakness, loss of libido and hormonal effects associated with interruption of the testosterone cycle. This therapy can have only a modest effect on BPH symptoms and the flow rate of urine. In addition, anti-androgens, such as 5-alpha-reductase, require months of therapy before LUTS improvements are observed. Surgical treatments of BPH carry a risk of complications including erectile dysfunction; retrograde ejaculation; urinary incontinence; complications related to anesthesia; damage to the penis or urethra, need for a repeat surgery, and the like. Even TURP, which is the gold standard in treatment of BPH, carries a high risk of complications. Adverse events associated with this procedure are reported to include retrograde ejaculation (65% of patients), post-operative irritation (15%), erectile dysfunction (10%), need for transfusion (8%), bladder neck constriction (7%), infection (6%), significant hematuria (6%), acute urinary retention (5%), need for secondary procedure (5%), and incontinence (3%). Typical recovery from TURP involves several days of inpatient hospital treatment with an indwelling urethral catheter, followed by several weeks in which obstructive symptoms are relieved, but there is pain or discomfort during micturition.

The reduction in the symptom score after minimally invasive procedures is not as large as the reduction in symptom score after TURP. Up to 25% of patients who receive these minimally invasive procedures ultimately undergo a TURP within 2 years. The improvement in the symptom score generally does not occur immediately after the procedure. For example, it takes an average of one month for a patient to notice improvement in symptoms after TUMT and 1.5 months to notice improvement after ILC. In fact, symptoms are typically worse for these therapies that heat or cook tissue, because of the swelling and necrosis that occurs in the initial weeks following the procedures. Prostatic stents often offer more immediate relief from obstruction but are now rarely used because of high adverse effect rates. Stents have the risk of migration from the original implant site (up to 12.5% of patients), encrustation (up to 27.5%), incontinence (up to 3%), and recurrent pain and discomfort. In published studies, these adverse effects necessitated 8% to 47% of stents to be explanted. Overgrowth of tissue through the stent and complex stent geometries has made their removal quite difficult and invasive.

Thus the most effective current methods of treating BPH carry a high risk of adverse effects. These methods and devices either require general or spinal anesthesia or have potential adverse effects that dictate that the procedures be performed in a surgical operating room, followed by a hospital stay for the patient. The methods of treating BPH that carry a lower risk of adverse effects are also associated with a lower reduction in the symptom score. While several of these procedures can be conducted with local analgesia in an office setting, the patient does not experience immediate relief and, in fact, often experiences worse symptoms for weeks after the procedure until the body begins to heal. Additionally, current device approaches require a urethral catheter placed in the bladder, in some cases for weeks. In some cases catheterization is indicated because the therapy actually causes obstruction during a period of time post operatively, and in other cases it is indicated because of post-operative bleeding and potentially occlusive clot formation. While drug therapies are easy to administer, the results are suboptimal; some drugs require significant time to take effect, and often entail undesired side effects.

Cosmetic or Reconstructive Tissue Lifting and Repositioning

Many cosmetic or reconstructive surgical procedures involve lifting, compressing or repositioning of natural tissue, natural tissue or artificial grafts, or aberrant tissue. For example, surgical procedures such as face lifts, brow lifts, neck lifts, tummy tucks, and the like, have become commonplace. In many cases, these procedures are performed by creating incisions through the skin, dissecting to a plane beneath muscles and fascia, freeing the muscles, fascia and overlying skin from underlying structures (e.g., bone or other muscles), lifting or repositioning the freed muscles, fascia and overlying skin, and then attaching the repositioned tissues to underlying or nearby structures (e.g., bone, periostium, other muscles) to hold the repositioned tissues in their new (e.g., lifted) position. In some cases, excess skin may also be removed during the procedure.

There have been attempts to develop minimally invasive devices and methods for cosmetic lifting and repositioning of tissues. For example, connector suspension lifts have been developed where one end of a standard or modified connector thread is attached to muscle and the other end is anchored to bone, periostium or another structure to lift and reposition the tissues as desired. Some of these connector suspension techniques have been performed through cannulas or needles inserted through relatively small incisions of puncture wounds.

Numerous existing surgical procedures are designed to treat urinary incontinence. The traditional surgical treatment for urinary incontinence is to add backboard support to the urethral posterior wall usually by repositioning the vagina with connectors. This significantly invasive procedure provides the backboard support needed for lumen closure during stress with concurrent pulling of the urethropelvic ligaments to prevent urine leakage. Another widely used therapy for incontinence is the placement of a sling that runs under the urethra and then either tethered to the transobterator foramen or pubic fascia. Over time the sling mesh can erode into the urethra, requiring cutting and/or removing the implanted mesh.

There remains a need for the development of new devices and methods that can be used for various procedures where it is desired to lift, compress, support or reposition tissues or organs within the body with less intra-operative trauma, less post-operative discomfort and/or shorter recovery times. Further, there is a need for an apparatus and related method which is easy and convenient to employ in an interventional procedure.

The disclosed embodiments address these and other needs.

### SUMMARY

Briefly and in general terms, the disclosed embodiments are directed towards anchor assemblies for positioning within a patient's body. In one approach, a curved anchor formed from elastic material is used. The anchor can include an internal bore running a longitudinal length of the anchor. The bore can be sized to receive a needle or other delivery component. The anchor further includes one or more of wing and fin structures extending radially from the anchor's body. In a specific embodiment, the anchor includes two wings which are joined to form a platform on a first side of the anchor. A fin can further be provided and positioned on an opposite side of the anchor. The wing and fin are formed of resilient material such that during advancement to an interventional site, the wing and fin are compressed against the anchor body by a delivery sheath to thereby define a small profile well suited for atraumatic insertion into body tissue. When unconstrained, the wing and fin project away from the anchor body thus defining a large cross-section for effective tissue apposition.

In other embodiments, the anchor can define a generally straight longitudinal profile and includes one or more of wings and a fin. It is also contemplated that a connector be connected to the anchor. The connector can be strung through holes provided in the anchor platform or can be threaded through the anchor bore, or both. The connector can further be affixed to the anchor or the anchor can be configured to slide with regard to the connector. The connector can also be an integral part of the anchor. In one embodiment the integral connector can be thin at the point of joining the anchor so as to allow the anchor to easily change orientation with respect to the connector. In another embodiment the connector can have a preshaped orientation to the anchor, such that when not constrained by a delivery means, the anchor moves to a predetermined orientation to the connector.

One application of the present disclosure relates to tissue approximation. In particular, partial thickness suturing can be achieved using the disclosed approaches. The disclosed anchors are designed for tissue penetration, rotation within the tissue and providing anchoring strength.

Moreover, it is contemplated that the anchor can embody wings or fins formed from resilient wires. The anchor can further include a plurality of fins and two or more wings. The anchor can be constructed partially or completely of absorbable materials. Further, the anchor can be equipped with mesh structure designed to remain in a patient's body accomplishing desired tissue manipulation after the anchor body is absorbed. Additionally, the anchor can be equipped with structure such as radiopaque strips for remotely viewing the anchor positioning after implantation. Additionally the anchor and/or connector can be pre-loaded with medication or other compounds that elute over time. It is also anticipated that if the anchor is made of absorbable material, compounds may elute as the anchor is absorbed. Compounds may be designed to facilitate scarring or proliferation of connective tissue. Other compounds may be therapeutic, such as androgens, testosterone cycle inhibitors, etc.

Various apparatus for delivering the disclosed anchors is also contemplated. The apparatus can be configured to deliver and implant single or multiple anchors. Further, the delivery apparatus can embody structure intended to register the anchor in a number of particular orientations for implantation. Moreover, the anchors can embody flexibility gradients along a longitudinal length of their bodies to facilitate rotation of the anchors to form a T-bar in response to an applied tension.

Other features and advantages will become apparent from the following detailed description, taken in conjunction with the accompanying drawings, which illustrate by way of example, the features of the various embodiments.

### BRIEF DESCRIPTION OF THE DRAWINGS

FIG. 1A is a partial cross-sectional side view, depicting an anchor with folding wings and fin housed within a delivery apparatus;

FIG. 1B is a cross-sectional view, depicting the anchor of FIG. 1A;

FIG. 2A is a perspective view, depicting the anchor of FIG. 1 unconstrained;

FIGS. 2B-2C are a perspective view, depicting an alternate approaches to an anchor;

FIG. 3 is a perspective view, depicting the anchor of FIG. 2 with a connector attached thereto;

FIG. 4 is a perspective view, depicting another anchor embodiment with a connector attached thereto;

FIG. 5 is a perspective view, depicting an anchor with a generally straight body;

FIG. 6A is a partial cross-sectional side view, depicting an anchor having wings and a fin defined by wire forms and retained in a delivery apparatus;

FIG. 6B is a cross-sectional view, depicting the anchor of FIG. 6A;

FIG. 6C is a perspective view, depicting the anchor of FIG. 6 removed from the delivery apparatus;

FIG. 7A is a side view, depicting an anchor including a plurality of wires defining wings;

FIG. 7B is a partial cross-sectional side view, depicting the anchor of FIG. 7A within a delivery assembly;

FIG. 7C is a cross-sectional view, depicting the area of FIG. 7A;

FIG. 8 is a perspective view, depicting an anchor with multiple fin structures;

FIG. 9 is a perspective view, depicting an anchor with four fin structures;

FIG. 10 is a perspective view, depicting an anchor with a plurality of wing structures;

FIG. 11A is a perspective view, depicting an anchor with a mesh patch;

FIGS. 11B-11C are perspective views, depicting another approach to an anchor with an oversized mesh patch;

FIG. 12 is a perspective view, depicting an anchor with a longitudinal radiopaque marker;

FIG. 13 is a perspective view, depicting an anchor with a ring marker;

FIG. 14A is a side view, depicting a plurality of anchors housed within a delivery apparatus being delivered within tissue;

FIGS. 14B-14C are schematic views, depicting profiles of straightened and curved anchor structure;

FIG. 15A is a perspective view, depicting the plurality of anchors of FIG. 14 delivered within tissue prior to turning in the tissue;

FIG. 15B is a perspective view, depicting the anchors of FIG. 15A assuming a rotated position;

FIGS. 15C-15F are perspective views, depicting an alternate approach to deploying multiple anchors and structure for capturing proximal terminal ends;

FIG. 16 is an enlarged view, depicting a proximal portion of an anchor depicted in FIG. 15;

FIG. 17A is a perspective view, depicting an anchor including a slot for engaging a delivery apparatus;

FIGS. 17B-17C are perspective and cross-sectional views, depicting an alternative needle design;

FIGS. 17D-17F are perspective views, depicting an approach to suture routing;

FIGS. 18A-18F are perspective cross-sectional views and side views, depicting an alternate approach to anchor structure;

FIGS. 19A-29 are perspective views, depicting yet further approaches to anchor structure;

FIGS. 30A-30D are cross-sectional and perspective views, depicting treating urinary incontinence; and

FIGS. 31A-31B are perspective views, depicting another treatment application.

### DETAILED DESCRIPTION

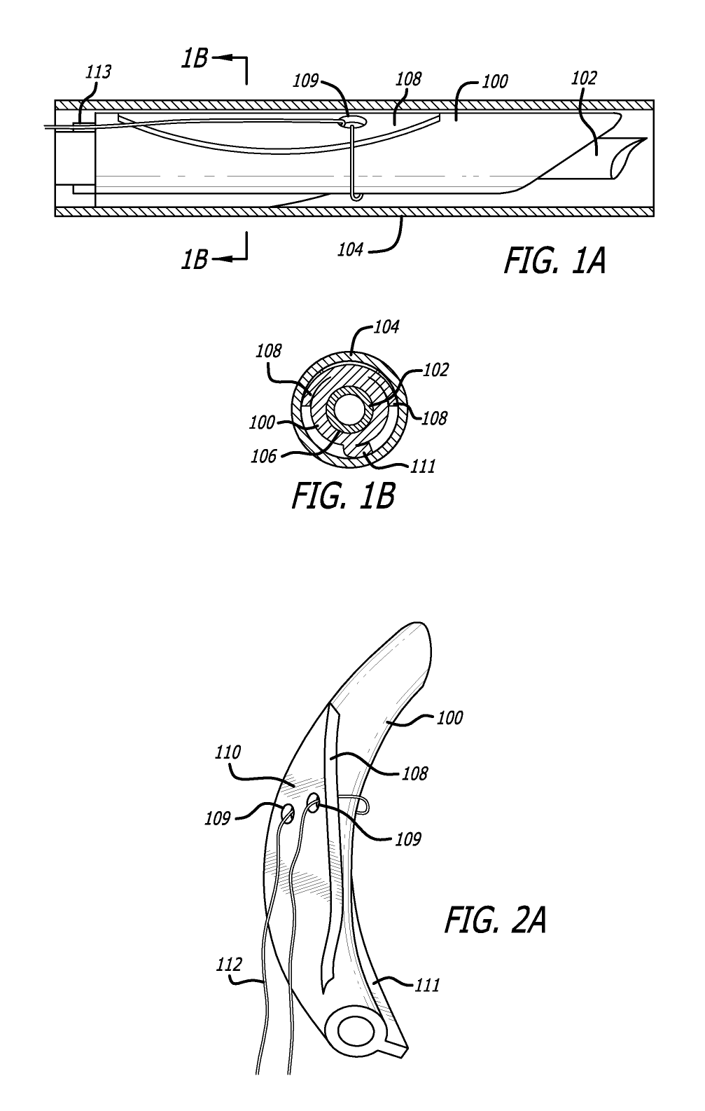

Turning now to the figures, which are provided by way of example and not limitation, the disclosed embodiments are embodied in anchor assemblies configured to be delivered within a patient's body. As stated, the disclosed embodiments can be employed for various medical purposes including but not limited to retracting, lifting, compressing, supporting or repositioning tissues, organs, anatomical structures, grafts or other material found within a patient's body. Such tissue manipulation is intended to facilitate the treatment of diseases or disorders. Moreover, the disclosed embodiments have applications in cosmetic, therapeutic, or reconstruction purposes, or in areas relating to the development or research of medical treatments. Referring now to the drawings, wherein like reference numerals denote like or corresponding components throughout the drawings and, more particularly to FIGS. 1A-31B, there are shown various embodiments of anchor assemblies.

In certain medical applications, one portion of an anchor assembly is positioned and implanted against a first section of anatomy. A second portion of the anchor assembly is then positioned and implanted adjacent to a second section of anatomy for the purpose of retracting, lifting, compressing, supporting or repositioning the second section of anatomy with respect to the first section of anatomy, as well as for the purpose of retracting, lifting, compressing, supporting or repositioning the first section of anatomy with respect to the second section of anatomy. Also, both a first and second portion of the anchor assembly can be configured to accomplish the desired retracting, lifting, compressing, supporting or repositioning of anatomy due to tension supplied thereto via a connector assembly (e.g., connector) affixed to the first and second portions of the anchor assembly.

In one embodiment of the anchor assembly, the anchor assembly is configured to include structure that is capable of being implanted within a patient's body. The anchor assembly can also be used in conjunction with a conventional remote viewing device (e.g., an endoscope) so that an interventional site can be observed.

In one specific, non-limiting application of the present disclosure is for the treatment of Benign Prostatic Hyperplasia. In this procedure, an implant is delivered into or through a prostatic lobe that is obstructing the urethral opening and restricting flow. The implant holds the lobe in a compressed state, thereby increasing the urethral opening and reducing the fluid obstruction through the prostatic urethra. In another embodiment the delivery instrument compresses the lobe and the anchor then fixes the lobe into the new geometry. The lobe tissue is not held under constant tension but is merely fixed in a smaller dimension by reducing the size of glandular ducts and/or blood vessels.

Another specific, non-limiting application relates to treating female urinary incontinence, preferably type II, due to urethral hypermobility. By way of background, the urine leaking process in type II incontinence starts with the anatomic support of the bladder neck weakening or the bladder shifting thus the proximal urethra gets displaced out of the abdominal pressure zone. Subsequently, when abdominal pressure, such as from sneezing, compresses the bladder, the urethra is not compressed. Therefore, the uncompressed urethra remains open and urine leaks out. In the treatment methods of the present application, an implant(s) is delivered to shift the bladder and/or bladder neck to offset intra-abdominal pressure or to change the profile or position of the urethra itself, to thereby decrease or eliminate incontinence. Another specific, non-limiting application relates to treating urinary incontinence, preferably type III, due to intrinsic sphincter deficiency. In the treatment methods of the present application, an implant(s) is delivered to the peri-urethral tissue. By compressing the tissue to become more firm or to extrude toward the urethra, the peri-urethral tissue can effectively become more supportive to the intrinsic sphincter function. Because no foreign material is directly supporting the urethra, the potential issue of erosion into the urethra (e.g. sling mesh material) is avoided.

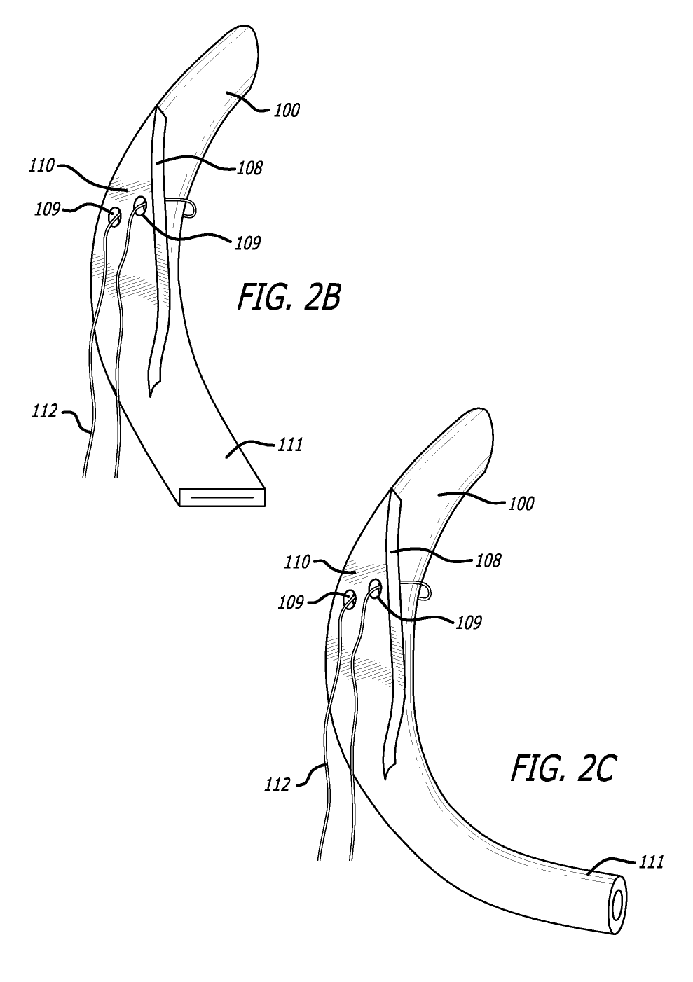

In one embodiment (FIGS. 1-2), the anchor 100 is sized to be configured about a needle 102 and to be received in a generally tubular delivery sheath assembly 104. As detailed below the anchor can be used independently or can form a part of an anchor assembly. In one approach the anchor defines a distal component of the anchor assembly.

The anchor 100 has a curved body and is formed from an elastic material such as silicone, polyethylene, PET, or Nylon. As detailed below, the curved body facilitates desired turning of the anchor within tissue upon the application of a tension force to the connector. An internal bore 106 extends a length of the anchor 100, the bore 106 being sized to receive the needle 102 during delivery of the anchor 100 to an interventional site. The anchor 100 further includes resilient or elastic wings 108 which define a platform 110 on a convex side of an unconstrained anchor body (See FIG. 2). The elastic wings are an important element of this anchor because they facilitate the anchor being reduced to a compressed size or folded for delivery inside of the delivery sheath 104. Of particular interest is a device including wings having a width less than or equal to one half of the anchor body circumference so that there is no overlap and for optimal packing. In this delivery configuration, the anchor can be delivered through tissue in a patient to a target site while creating a small profile delivery tract in the tissue, thus causing minimal tissue damage. In this regard, in its assembled form, the anchor 100 and delivery sheath 104 define a profile designed to impart a low force with minimal disruption to tissue through which the assembly is advanced. By creating a relatively smaller entry path, there is less of a possibility that a delivered anchor 100 will back out through the entry path. Moreover, since the anchor 100 includes wings 108 which resiliently unfold upon disengagement of the anchor 100 from the delivery assembly 104, the delivered anchor 100 timely and advantageously provides relatively larger, flat surfaces for approximating tissue. Thus, the anchor has a deployable profile close to the delivery tool and also a platform with sufficient surface area for applying forces to tissue. In the contemplated embodiments, the structure of the width aspect of the anchor is configured to define its full dimension prior or subsequent to the turning of the anchor in response to a tension.

Two holes 109 are formed in the platform 110 through which a connector, (e.g., suture, thread or wire) 112 is threaded. In one approach, the connector 112 is looped about the anchor body and is provided for manipulating the anchor 100 and providing a tension thereto. Since the connector 112 is not affixed to the anchor 100, it can slide freely and be an aid in certain aspects of tissue manipulation. To minimize or eliminate the risk of bacterial wicking, such as when treating benign prostatic hyperplasia, stress urinary incontinence or vaginal prolapse, it is preferable to use a monofilament suture as the connector. It is also contemplated that the suture can define a braid with a sleeve and/or an antimicrobial coating. The anchor 100 can additionally include an elastic or resilient fin 111 configured on an opposite of the anchor body from the platform 110. The elastic fin is an important element of this anchor similar to the elastic wings. In particular, the fin can act as a rudder during anchor turning and implantation thereby directing and guiding the anchor to a desired position.

The connector or other structural aspects can also be treated or impregnated with substances or coatings designed to reduce bacterial colonization or migration. In particular, the connector can be coated with materials such as silver or other antibiotic preparations. Further, the device can be treated with chemotherapeutic agents, anti-vascular agents, anti-androgenic agents, anti-cholingeric agents, alpha-blocking agents, analgesic, or other medication classes. In addition, radio-active agents or substances can be incorporated into the structure for selective tissue destruction. It is also contemplated that a dissolvable anchor can be employed so that fibrotic tissue is created in the ghost of the anchor thus forming a type of bioanchor. The devices can additionally be textured or treated to promote tissue ingrowth.

As can be appreciated from FIG. 1, when placed on the needle 102, the anchor 100 is straightened longitudinally to a generally straight configuration. The needle 102 can further include external structures for registering the anchor 100 on the needle 102 during advancement of the assembly to the interventional site. In this regard, the needle 102 can include structure 113 defining an enlarged section for registering the anchor 100. Additionally, the structure 113 can form a sleeve which is slideable over the needle 102 for advancing the anchor 100 beyond the needle or selectively positioning the anchor thereupon. In one embodiment the sleeve could be held in position while the needle is retracted so as to free the anchor from the needle or trocar.

As shown in FIG. 2A, the wings 108 and fin 111 are angled to facilitate tissue penetration and approximation. A proximal portion of the fin 111 is designed to resist dislodgement or pull-out of the anchor 100 after delivery of the anchor 100 at the target tissue and can help engagement of the anchor with tissue and thus desired removal from the needle during deployment. The fin can also be a pre-shaped form of the proximal portion of the anchor, such that when the needle is removed from the core of the anchor, the proximal portion assumes the pre-shaped form of a vertically flattened fin or bluntly curved tail (See FIGS. 2A and 2B). The platform 110 can generally define an elliptical structure comprised of two wings 108 as seen in FIG. 2 which is tapered at opposite longitudinal ends. It is to be recognized that the sheath 104 is employed to retain the wings 108 and fin 111 in a compressed or restrained configuration during the delivery process, being withdrawn when the anchor 100 is positioned at a final implantation site. Alternatively, this tapered structure of the wings 108 can help facilitate tissue penetrating as the needle 102 is advanced within patient body anatomy in the event the sheath 104 is removed proximally during anchor 100 delivery and prior to its deployment at a desired implantable site. In any case, the wings 108 and fin 111 are permitted to unfurl or resiliently return to extended positions upon final placement or disengagement from the needle 102 and sheath 104, whether the anchor 100 is released from the needle 102 and sheath 104 simultaneously or individually in series. A lubricious film such as sodium stearate may be applied to the surface of the anchor to prevent adhesion to itself or the needle during storage in the folded configuration.

By applying tension to the connector 112, the deployed anchor 100 is rotated and secured against body tissue. As stated, the anchor 100 can assume a longitudinally curved shape after deployment from the delivery apparatus. This curved shape as well as the dynamic return to the curved profile also facilitates rotation of the anchor 100 when tension is placed thereon by the connector 112. Such turning of the anchor can be key to achieving anchoring in tissue as the turned anchor 100 presents a significant structure generally perpendicular (such as a T-bar configuration) to the direction of tension being applied by connector 112.

When tension is applied to the connector 112 attached to an anchor 100, the fin 111 guides the rotation of the anchor and can prevent the anchor 100 from twisting or moving in an undesirable fashion within tissue. Thus, the anchor 100 is positioned generally perpendicular to the connector 112 to a fastening position as are the wings. The dimensions of the wings 108 are also selected to help achieve proper rotation of the anchor in tissue in that the wings 108 can be positioned on the anchor body closer to a proximal end of the anchor than a distal end. This same objective is achieved with the position of the connector holes 109 along the anchor body. The anchor is repositioned from vertical to horizontal to resist pull-out.

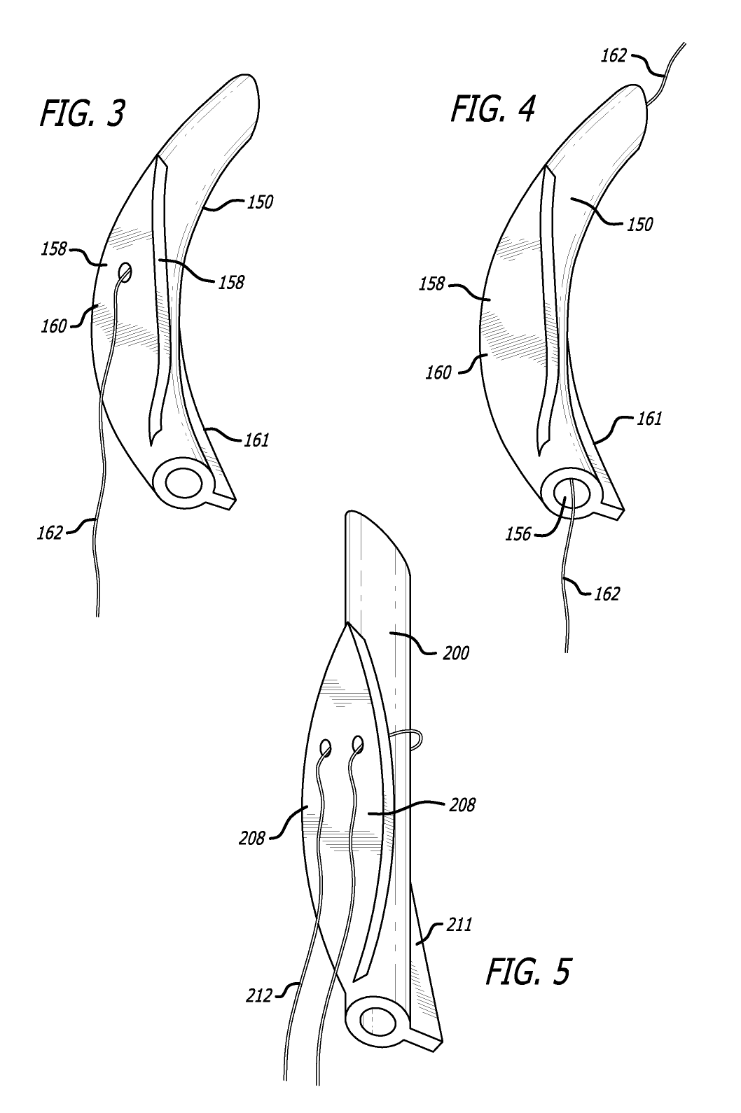

As shown in FIG. 3, an alternative approach is to fix one connector 162 at a midpoint of the anchor 150 and between wings 158 of an anchor 150. Such a configuration facilitates the turning of the anchor 150 upon application of a tension force. Furthermore, a single strand connection to the anchor 150 simplifies the assembly, eliminates the possibility of two or more connectors from becoming intertwined or bound during delivery and deployment. As before, the anchor includes a resiliently formed platform 160 and a fin 161 which are compressed during advancement to a surgical site and which extend outwardly upon deployment. Thus, the anchor/delivery sheath defines a relatively small profile to minimize tissue disruption during advancement to an interventional site and upon deployment, the anchor 150 defines a large surface area for tissue approximation. The affixed connector 162 can in certain approaches provide a tactile feel which assists a physician in desired anchor placement. As shown in FIG. 4, connector 162 can also be affixed within an internal bore 156 of an anchor 150 body and looped longitudinally thereabout (not shown). In this way, rotation of the anchor 150 can be accomplished essentially by applying forces at opposite ends of the anchor 150. This anchor 150 can also be rotated longitudinally with respect to the connector 162 so that the platform can be placed as desired against tissue.

It is also contemplated that any of the disclosed anchors can have a generally straight unconstrained body, such as the anchor 200 shown in FIG. 5. Thus, while the elastic wings 208 and elastic fin 211 will be constrained during anchor advancement within tissue, such anchors will have a body which remains relatively straightened. Accordingly, the generally straight anchor 200 can be deployed more easily from within a sheath or off a needle as frictional forces and other stresses between the anchor 200 and delivery components are minimized. It is also contemplated that the anchor material can be lubricious or coated with lubricious material so as to facilitate delivery off of the needle.

In the disclosed anchor embodiments, a large, fully expanded profile is presented after anchor implantation. Such a fully expanded profile can either be presented immediately upon release from a delivery system, or the anchor can be configured to define its full dimension before or after its turning against tissue. Thus, the full extent of the anchors can be achieved independently through self-expansion or in response to a tension applied to a suture attached to the anchor.

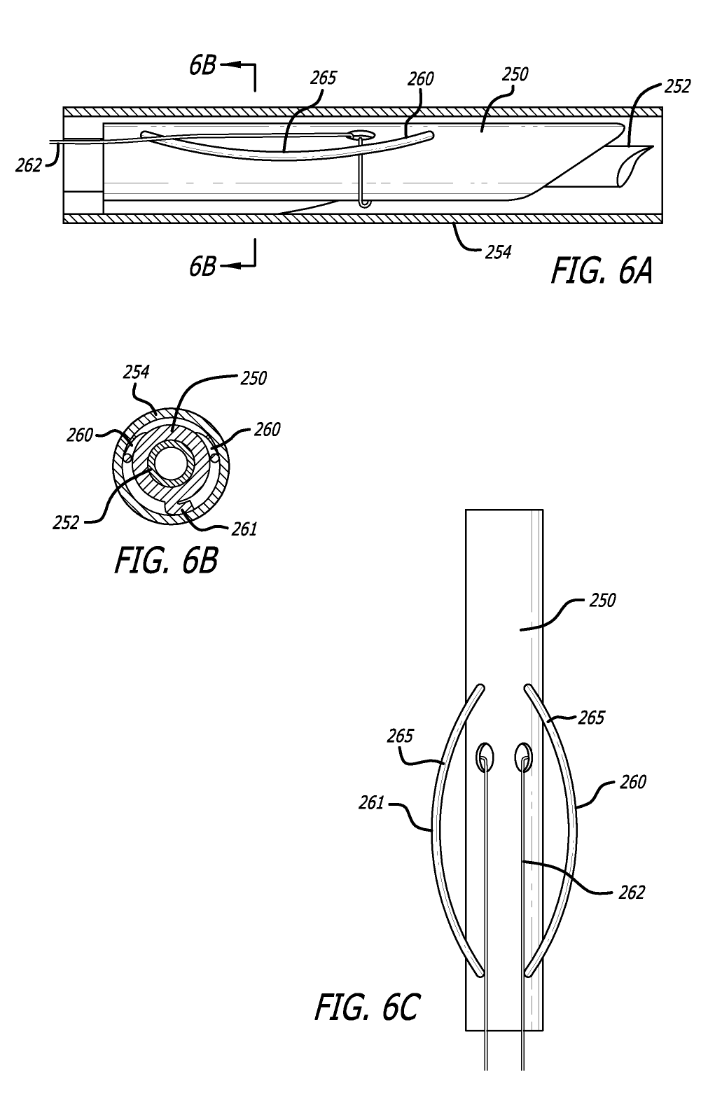

Other structure defining wings and fins can be incorporated into an anchor as well. As shown in FIGS. 6A-6C, an anchor 250 can include wings 260 and a fin 261 defined by or including loops of a wire 265. The wire loops 265 are folded against the anchor body during advancement while carried by a delivery apparatus including a needle 252 and a retaining sheath 254. Such loops 265 also advantageously define lateral dimensions suited for applying approximation forces after implantation, while also tucking away nicely to help define a small insertion profile to minimize tissue trauma. The looped structure may also provide a space for tissue in growth to facilitate a permanent connection within tissue. It is contemplated that these wire loops could be webbed with thin elastic or inelastic material. One embodiment is a porous mesh that further allows for tissue ingrowth.

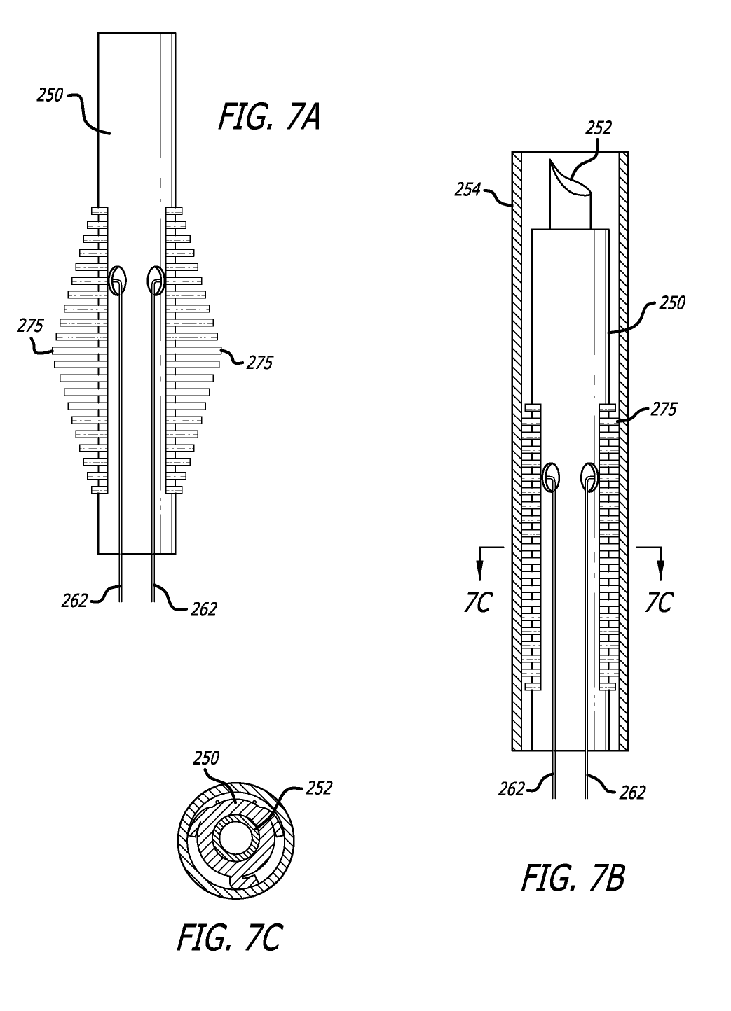

The anchor 250 can alternatively include (See FIG. 7A-B) wire-forms 275 which make up the wings 260 and fin (not shown) and can be integral to the anchor body or be defined by individual wires attached as an assembly. The number of wire forms can be chosen to be just sufficient enough to support forces expected to be applied thereto and can be formed from stainless steel, Nitinol or a polymer and/or be overmolded with a silicone. The structure can thus be as light weight and easy to manipulate as possible while providing a platform for desired tissue approximation. Again, these wire-forms 275 are folded against the anchor body to define an atraumatic insertion profile. Here, as shown, the wires-forms 275 can be folded radially. Alternatively, the wire-forms 275 can be folded proximally or distally initially and permitted to extend laterally upon anchor deployment. The wire forms 275 define the desired width component while also providing a high surface area for tissue in growth.

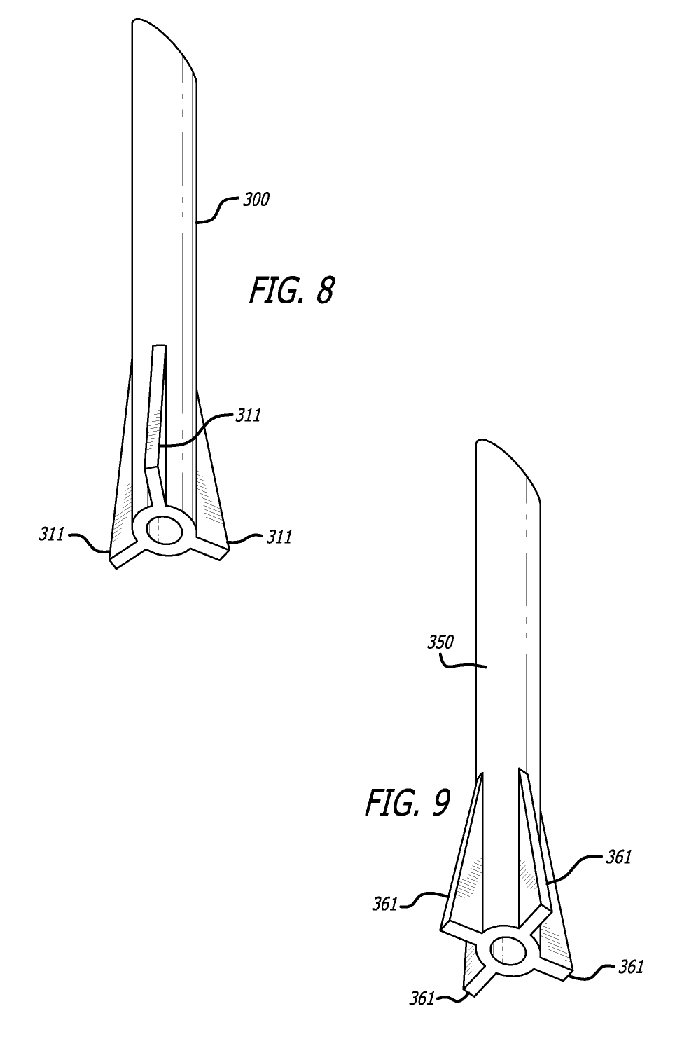

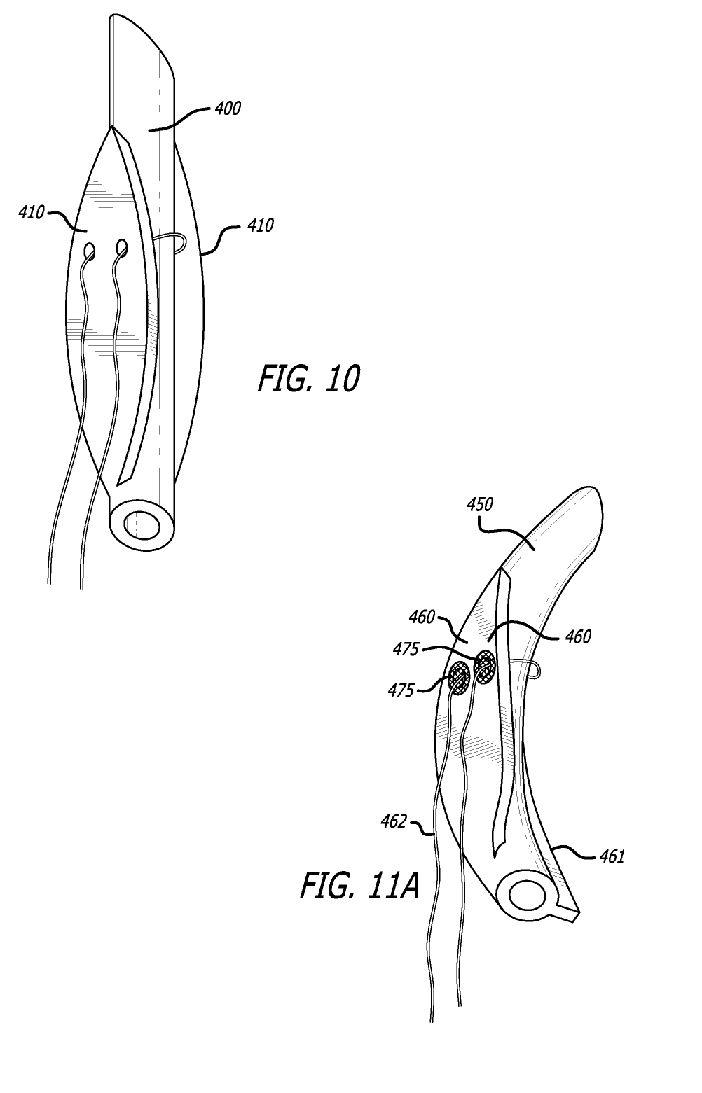

Other, alternative forms of elastic wings and elastic fins are encompassed by the present invention. An anchor can have three wings. As shown in FIG. 8, an anchor 300 can include multiple fin structures 311. An anchor 350 with a four-fin 361 configuration is also contemplated (See FIG. 9) as is an anchor 400 with multiple elastic wing structures 410 (See FIG. 10). The plurality of fins and wings increase the surface area and possible angles of structures for accomplishing tissue approximation. Additionally, more bearing surfaces are presented to capture tissue. Further, the surface of any structure can be treated by processing methods such as sand blasting or the like to increase microscopic and macroscopic roughness. The roughened bearing surfaces are presented to encourage tissue in growth and affixation.

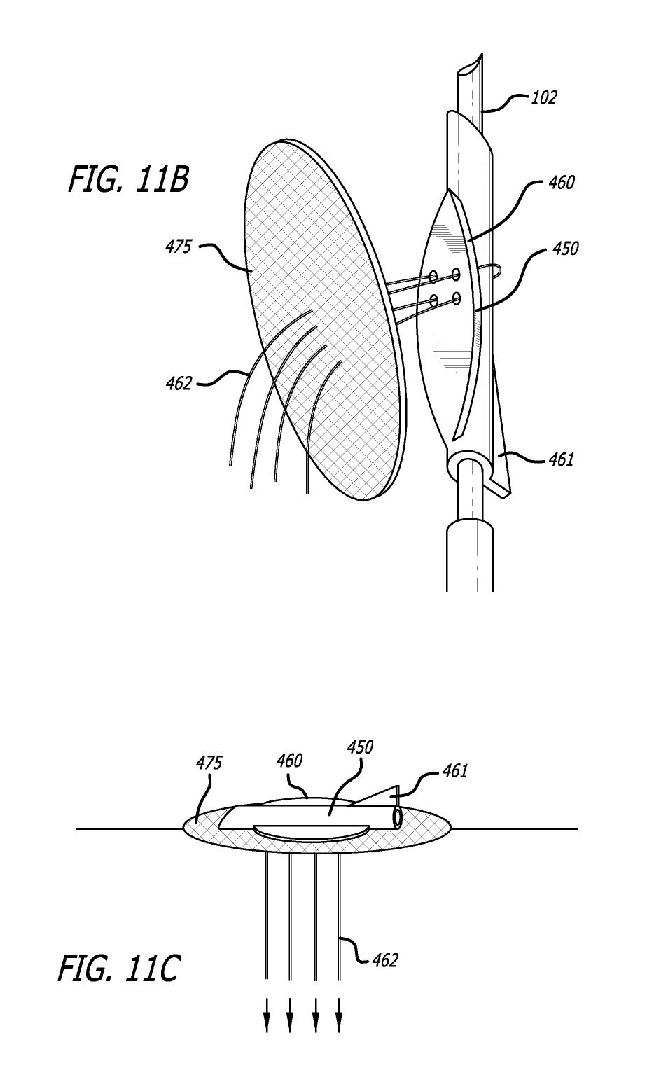

Further, as shown in FIG. 11A, an anchor 450 is equipped with one or more mesh patches 475 which is arranged between the anchor 450 and proximal sections of a connector 462. In certain situations, the anchor body 450, mesh patch, or connector can be formed of a bio-absorbable material. The mesh patches 475 can be intended to remain in a patient's body attached to the connector 462 after the anchor body has dissolved. The mesh patch can take on myriad shapes and configurations which aid in securing the apparatus at an interventional site because of tissue in-growth into the mesh. That is, the mesh can facilitate chronic tissue in growth and creation of a fibrotic matrix for securely holding the device in place. For example, as shown in FIGS. 11B and 11C, the mesh patch 475 can have a width and a length which encompasses that of the anchor 450. Thus, once the anchor body 450 dissolves, a sufficiently large mesh surface area remains to accomplish tissue approximation. The bio-absorbable structures can also have rough or jagged surface features to promote tissue ingrowth or hasten body resorption.

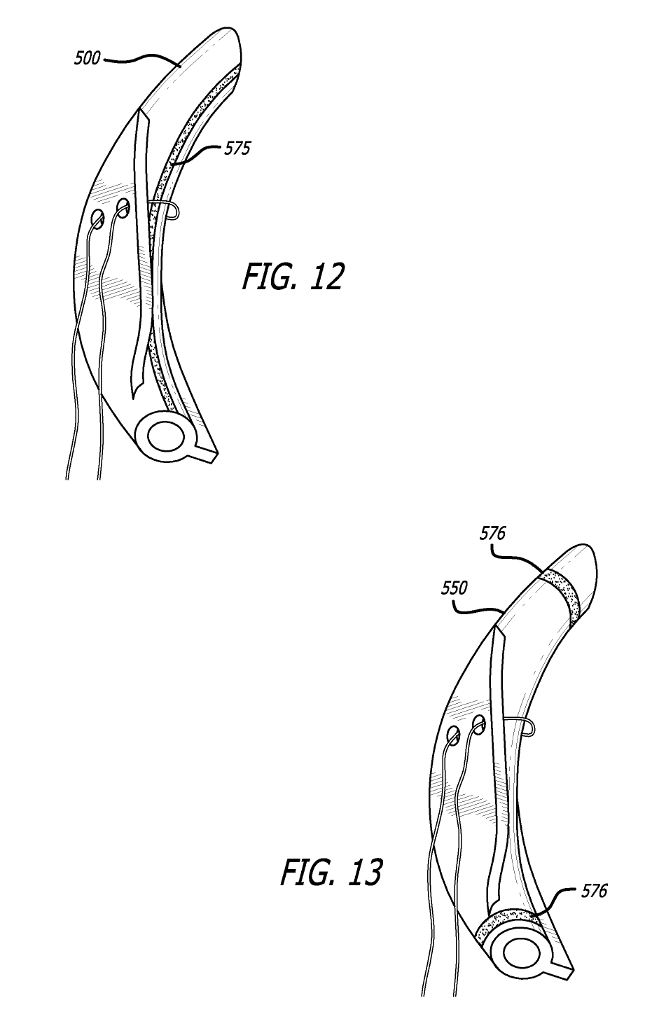

In some procedures it is advantageous to be able to remotely identify the position of an anchor during advancement to a surgical site and/or subsequent to its implantation. In this regard, fluoroscopy or other remote imaging techniques can be employed. To accomplish this, one or more of the disclosed anchors can include radiopaque markers. The radiopaqueness can be incorporated by overmolding, via an assembled marker such as a platinum iridium band or wire or foil or small particulates molded into the anchor. One approach (FIG. 12) can involve providing an anchor 500 with a longitudinal marker 575 extending entirely or partially along a length of the anchor 500. One or more radiopaque rings 576 can alternatively or additionally be incorporated into an anchor body 550 (FIG. 13). The pattern of the radiopaque markings can be used to identify the orientation of the anchor in the patient's tissue. Therefore, relying on the markers, a physician can locate the position of the anchor during advancement to an interventional site. After implantation, proper positioning of the anchor can be confirmed and to identify whether there has been any movement of the device such as from vibrational energy. In addition, the radio-opaque marker could be made of a anti-bacterial substance such as silver which would address visualization during delivery as well as infection control after implantation. The radio-opaque marker could also be used as a method to determine the rate of bioabsorption via reduction in thickness. Alternatively, the radio-opaque markers could be used to provide positional information after implantation. Specifically, the marker could be used to monitor changes in tissue size. Bio-absorptive material could be specifically implanted which would allow parts of the anchor to separate. Subsequent movement of those anchors could be quantified through non-invasive assessment.

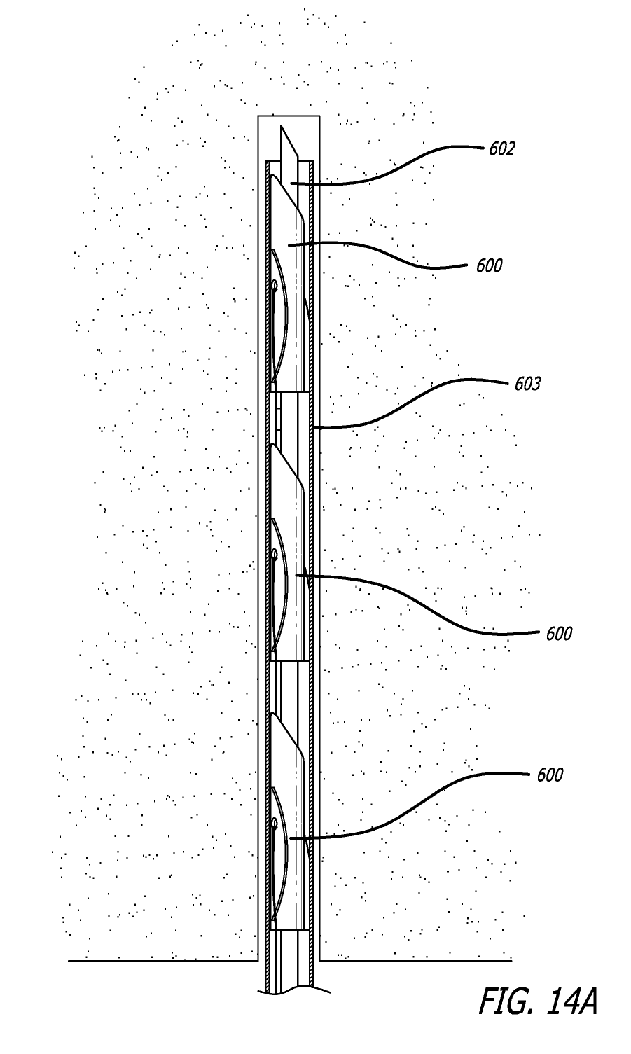

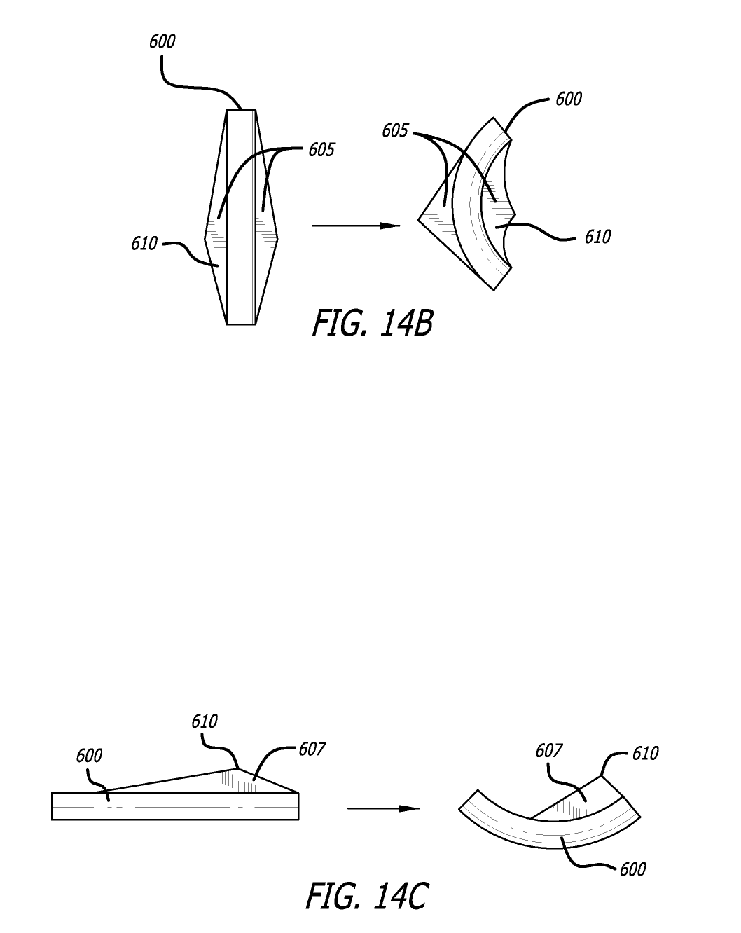

Turning now to FIGS. 14A-20, various other contemplated anchors including both desirable longitudinal and lateral components are presented. In one embodiment, it is further contemplated that a system involving delivery of a plurality of anchors can be employed to achieve desired tissue manipulation or approximation. A single common connector can be utilized to string the anchors together or a plurality of connectors can be employed. Such anchors can be inserted in-line within tissue and arranged so that the anchors are on one side of a tissue mass that is targeted for approximation. Alternatively, the multiple anchors can be placed within or through two or more tissue layers that are targeted for connection. It is further contemplated that the anchors can be deployed at various orientations with respect to each other so that certain anchors turn or flip in opposite directions and/or are rotated out of plane with respect to each other. In this regard, anchors can be deployed for example, at 90° or 180° differing rotational angles. It is further contemplated that tension can be applied to individual anchors separately in a predetermined order e.g. distal-most anchor first, so that a tension is applied to a first anchor while shielding a second anchor from tension. Additionally, tension can be applied simultaneously to a plurality of anchors in a particular application. It is further considered that the plurality of anchors can be achieved by varying the width, roughness, profile, or number of the connecting member alone. In this case, one can imagine that the anchoring system can be achieved by deploying a single material (such as suture material) into the tissue.

Further, since the suture can be configured so that it is not bound to one or more of the plurality of anchors, such anchors are free to move closer together in response to an applied tension. This can in certain circumstances provide an important versatility in approach, for example, where it is found in situ to be necessary to apply greater forces to targeted tissues.

With reference to FIGS. 14A-14C, there is depicted one approach for delivering multiple anchors 600 at a surgical site. A delivery apparatus including a needle 602 and outer sheath 603 can be configured to house two or more anchors 600 with elastic retaining wings 605 and elastic fins 611 of the anchors 600 in a restrained configuration prior to deployment. While held in a restrained configuration, the wings, tail and anchor body exhibit a reduced cross-sectional area to minimize tissue resistance when advancing the structure through tissue. Additionally, by virtue of being straightened longitudinally within the confines of the outer sheath 603 and about the needle 602 (See FIGS. 14B and 14C), the wings 605 and tail 607 of an anchor 600, whether folded or not, present a lower profile device for advancement through tissue. The wings 605 and tail 607 of the anchors 600 embody a hinge point 610 so that when straightened for advancement through tissue, the anchor is long in its straightened configuration and the wings 605 and tail 607 have a low profile. Upon deployment from the delivery apparatus, the anchor is shorter in its deployed, curved configuration and the wings 605 and tail 607 then open to a larger profile to thereby present a larger foot print for anchoring in the tissue.

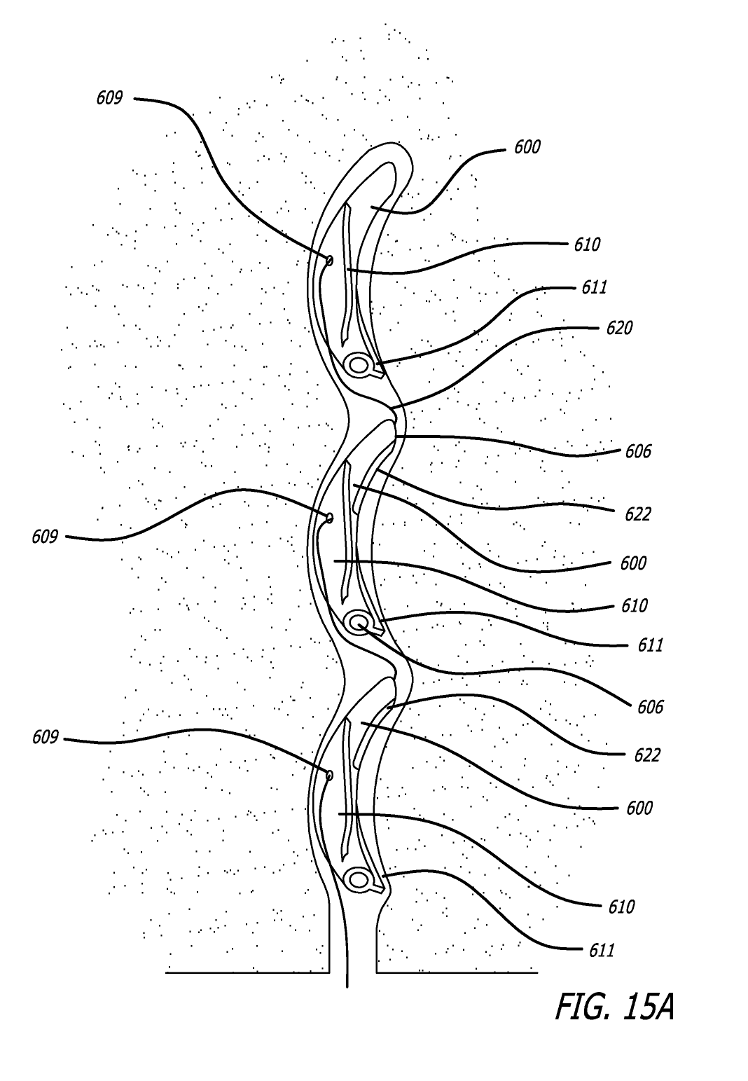

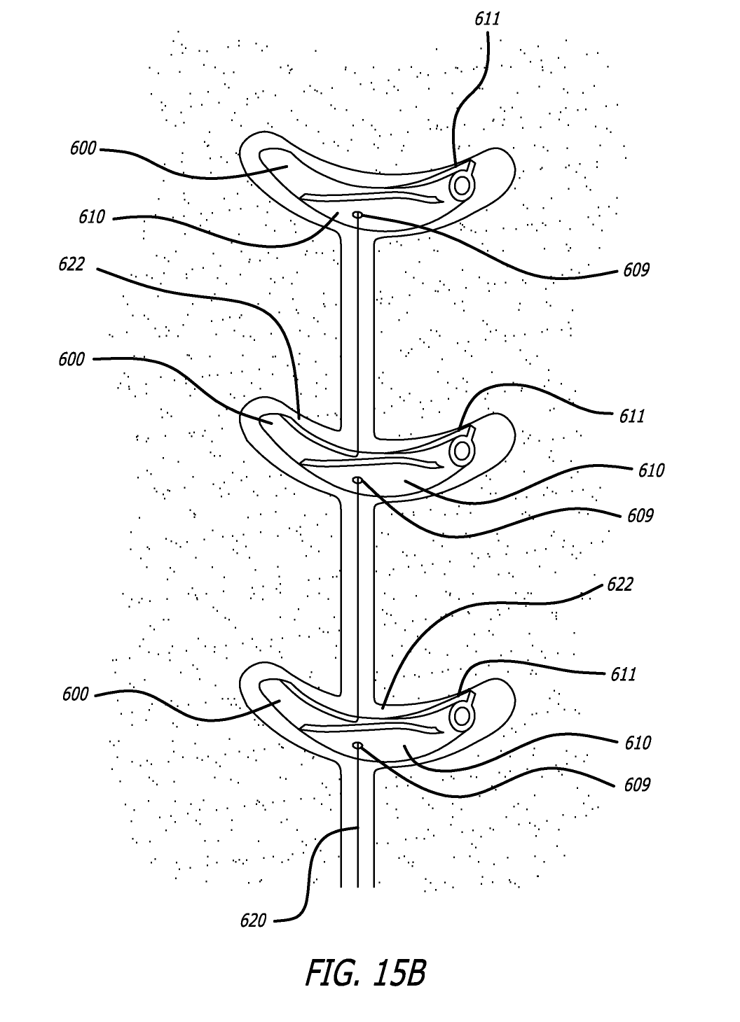

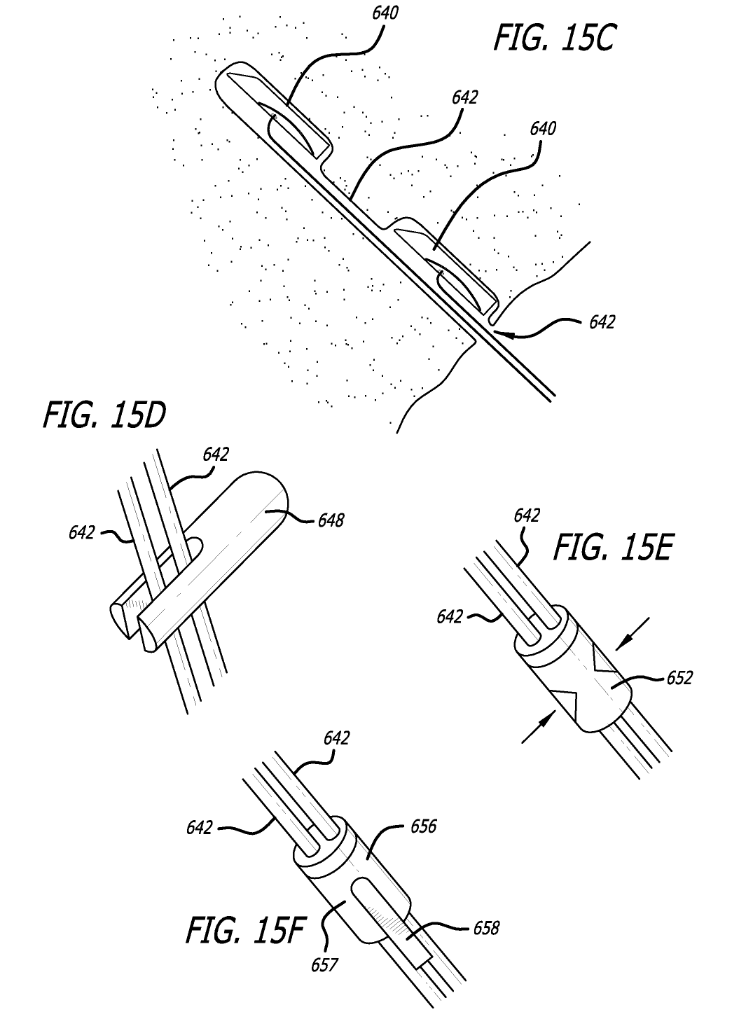

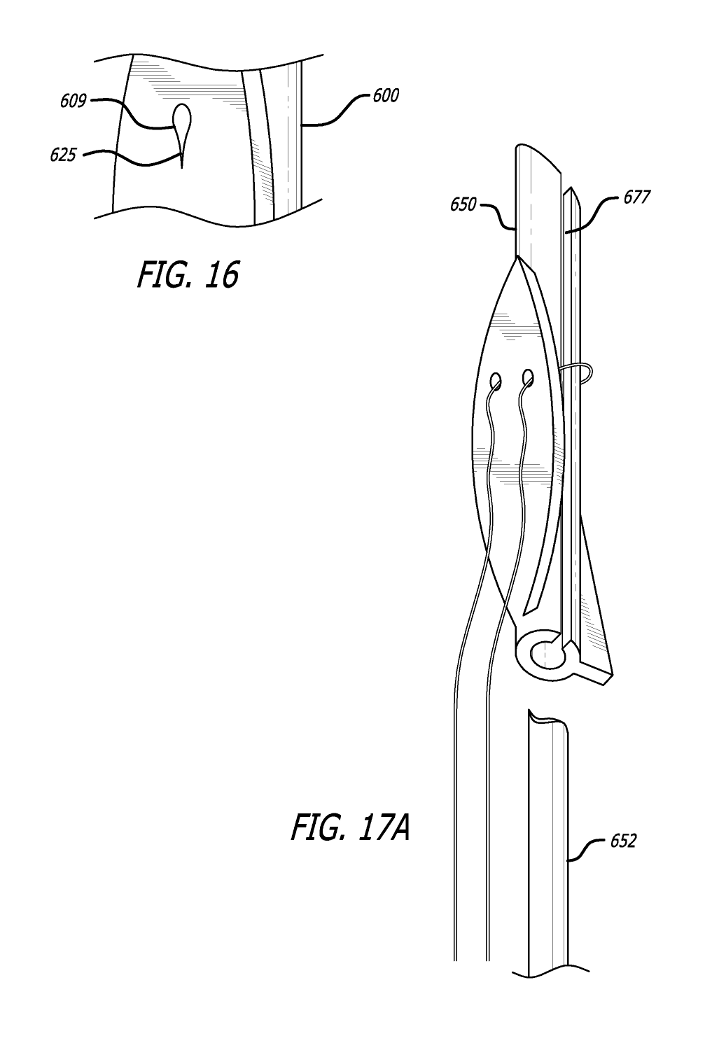

FIG. 15A depicts the various plurality of anchors deployed within tissue with the wings 610 and fins 611 assuming their curved configuration prior to rotation in response to tension applied to the anchors. Again, here, a single connector 620 can be utilized to provide tension to the anchors 600. The connector 620 can be affixed to a distal or lead anchor 600 and then threaded through and within an internal bore 606 of proximal adjacent anchors 600 and out a connector hole 609. Such pluralities of anchors can be individually deployed and turned prior to deploying a subsequent anchor or the anchors 600 can be successively deployed without accomplishing the turning of the anchors 600. Thus, tension can be shielded from one or more anchors while a particular anchor is implanted. Also, subsequent anchors may be partially exposed to prevent pull back into or onto the needle to thereby facilitate flipping. Alternatively, as stated, tension can be applied to all anchors simultaneously. According to facilitating such desired turning of the anchors 600, the connector holes 609 can be equipped with a slot 625 for gripping the connector 612 (See FIG. 16).

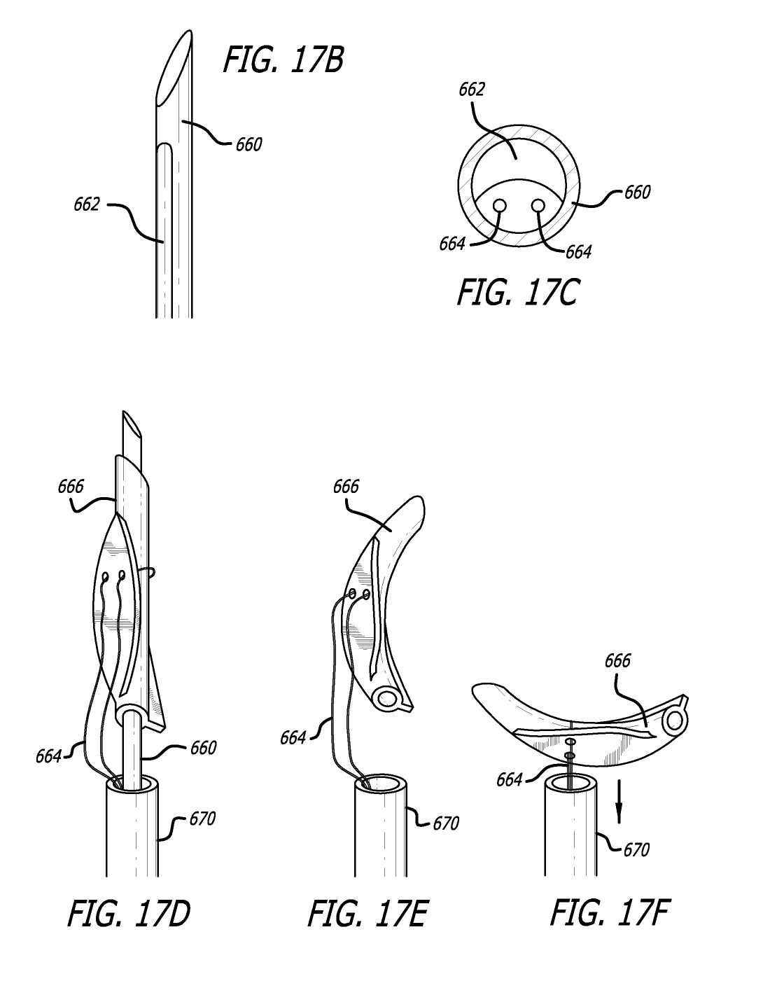

Various other anchor delivery apparatus are also contemplated. In one alternative approach, the needle 652 can include a longitudinally extending ridge or rail 675 formed on its exterior. The rail 675 can be sized and shaped to form a dove tail or T-bar connection with a corresponding slot 677 formed in one or more anchors 650. (See FIG. 17A) In certain applications, such an arrangement can aid in avoiding the unwanted rotation of an anchor 650 around the needle, thus providing better predictability in deployment and delivery of anchors within tissue.

Referring specifically to FIG. 15B, it is noted that upon applying a tension to the suture 620 can result in turning the anchors 600 within tissue. The tensioning of the suture 620 can occur after all of the anchors 600 are deployed at an interventional site or a deployment device can be configured to deliver a first anchor 600 and thereafter apply tension and then repeat the process after deploying each subsequent anchor 600. In order to facilitate rotation of the anchors 600 within tissue, anchors 600 positioned proximal to a lead anchor can include a slot 622 allowing the anchor 600 to rotate in response to tension while the suture 620 extends relatively straight to the next anchor.

In an alternative approach (FIG. 15C), a plurality of anchors 640 can be deployed at an interventional site individually, each anchor 640 including a suture 642 attached thereto. As before, the implantation of multiple anchors has a benefit of increased anchoring strength as each anchor can contribute to an overall holding function. Where this approach is taken, a proximal end of the suture 642 extending from the anchors 640 can be locked together in a manner to better control the multiple suture lengths.

In a first locking method (FIG. 15D), a clothespin-like device 648 sized and shaped to lockingly receive the sutures 642 can be employed. In this regard, a press fit is accomplished between an opening defined by prongs of the clothespin-like device 648 and the sutures 642. A second approach to locking the sutures 642 involves a generally tubular device 652 (FIG. 15E) which has a deformable mid-section. Here, the mid-section of the tubular device 652 can be crimped to accomplish locking the sutures 642 in place. In yet another approach, an interference fit between the sutures 642 can be achieved with a two part locking device 656. A first piece 657 of the device, such as a tube, and a second piece 658 is sized and shaped to be received in the tube and to lock the sutures 642 relative thereto.

As shown in FIGS. 17B and 17C, a stylet 660 of an anchor delivery system can include concave structure 662 intended to facilitate effective routing of sutures 664 in the concave structure 662 so the sutures and stylet can remain within the diameter of a low profile pusher. In this way, an anchor can be disengaged from a needle 660 without interference from the suture 664 and a needle 660 can be better manipulated with respect to other structure such as an outer sheath of a delivery system.

With reference to FIGS. 17D-17F, an anchor 666 configured about a needle 660 can be positioned or advanced beyond a tubular pusher device 670. The needle 660 is then withdrawn while maintaining the pusher 670 in place (FIG. 17E). Tension can then be applied to the sutures 664 to affect the turning of the anchor 666 as desired or against target tissue. Routing suture through the pusher device 670 creates a fulcrum point where the anchor rotates as tension is applied to the suture. This simple approach to anchor advancement and deployment is improved by the stylet 660 configured with the concave structure 662 which aids in effectively routing the sutures 664.

Turning now to FIGS. 18A-18F, there is shown another embodiment of an anchor 680. The anchor 680 can be formed from a polymer and can be a permanent structure or can be absorbable. The device includes a body 682 and a pair of fulcrums 683 projecting laterally from the body 682. The fulcrums 683 are designed to engage tissue and provide a platform supporting the device against tissue. A suture 684 is fixed on a first side of the body 682 and extends through a hole 685 in the body at a midpoint thereof.

As tension is applied to the suture 684, the fulcrums 683 are held relatively stationary in tissue and the body 682 rotates as depicted in FIGS. 18C and 18D. A balance or desired imbalance of proximal end stiffness of the anchor 680 to fulcrum support allows the proximal portion to deflect under initial suture tension (See FIGS. 18C and 18E), which increases an off axis presentation of the proximal end of the anchor 680. Accordingly, the flexing of the proximal end operates to resist the backing out of the anchor 680 through the insertion path.

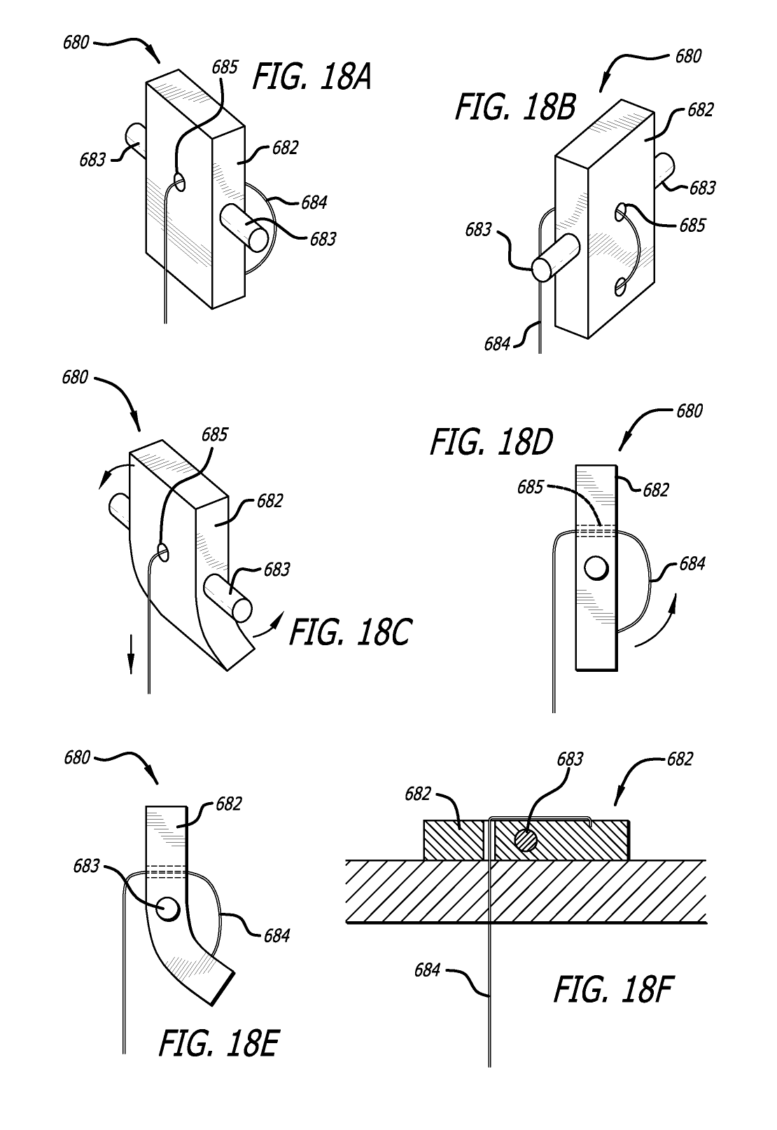

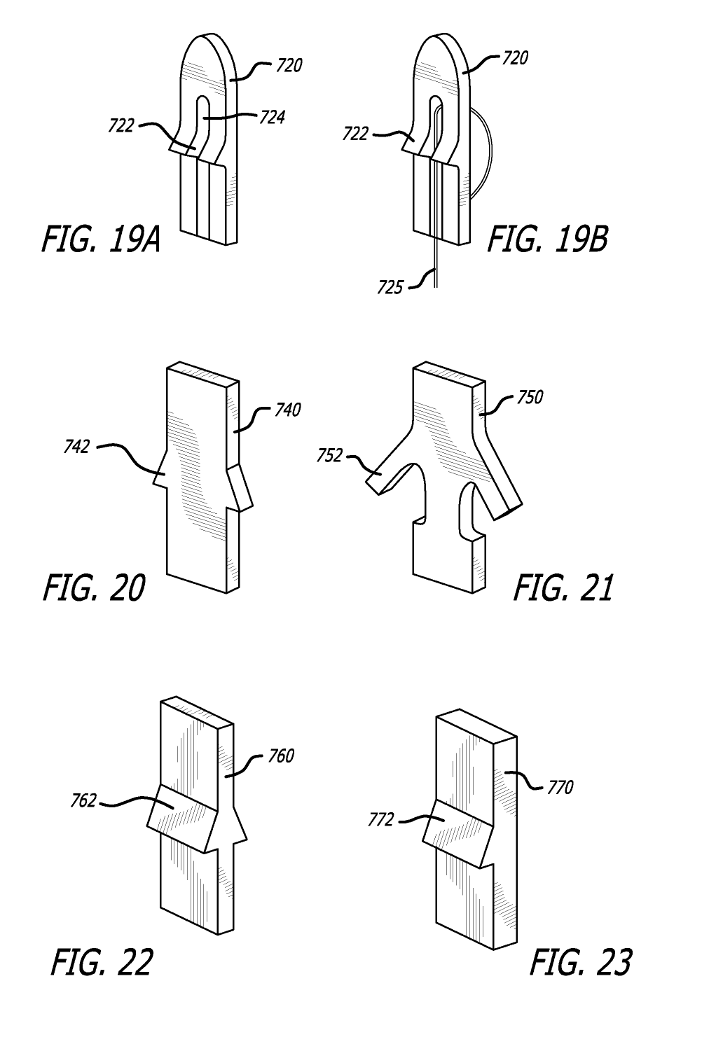

Certain other specific anchors embodying variations on fulcrum features are shown in FIGS. 19A-23. FIGS. 19A-19B depict an anchor 720 with turning fulcrums 722 configured along an underside surface of the anchor 720. A slot 724 can further be provided to accept a length of a suture 725 and exclude the suture from engagement with tissue. More laterally oriented fulcrums 742, 752, 762, 772 are formed on the anchors 740, 750, 760, 770 shown in FIGS. 20-23. In particular, the fulcrums 752 of the anchor 750 shown in FIG. 21 are unique in that they can be folded inwardly during advancement of the anchor 700 to a treatment site, later resiliently expanding to define a larger width and area for engaging tissue.

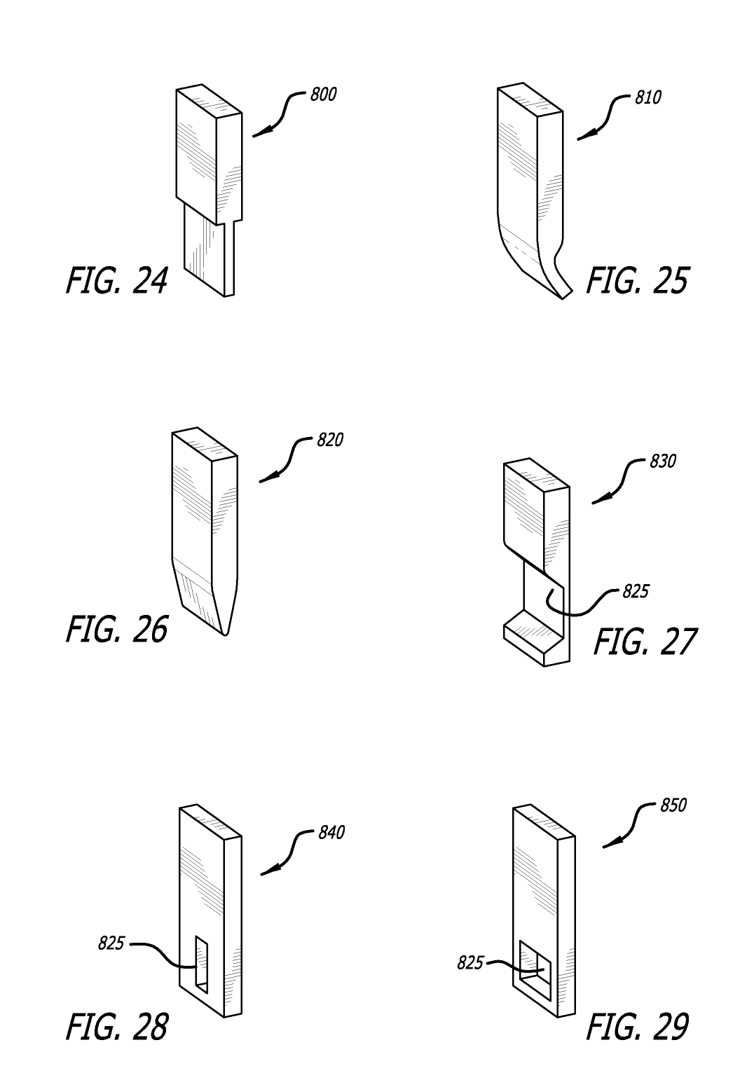

Also included are various cross-sectional shapes and longitudinal configuration for anchors so that desired proximal end flexibility is presented to accomplish turning of the anchor within tissue so as to avoid movement of the anchor proximally through a tissue insertion path (See FIGS. 24-29). Various anchors embody varying flexibility gradient profiles which are suited for use in specific areas of a patient's body. Thus, an anchor 800 having stepped profile provides a first flexibility along a distal or leading portion and a second flexibility along a proximal or trailing portion (FIG. 24). An anchor 810 (FIG. 28) can also include a narrowing tail portion that curves in the direction that the anchor is intended to rotate upon the application of a tension. Moreover, an anchor 820 (FIG. 26) can alternatively have a tapered, straight profile thus including a more flexible trailing end. Variously shaped cut-outs 825 can also be formed in anchors 830, 840, 850 to provide the devices with desirable flexibility gradients.

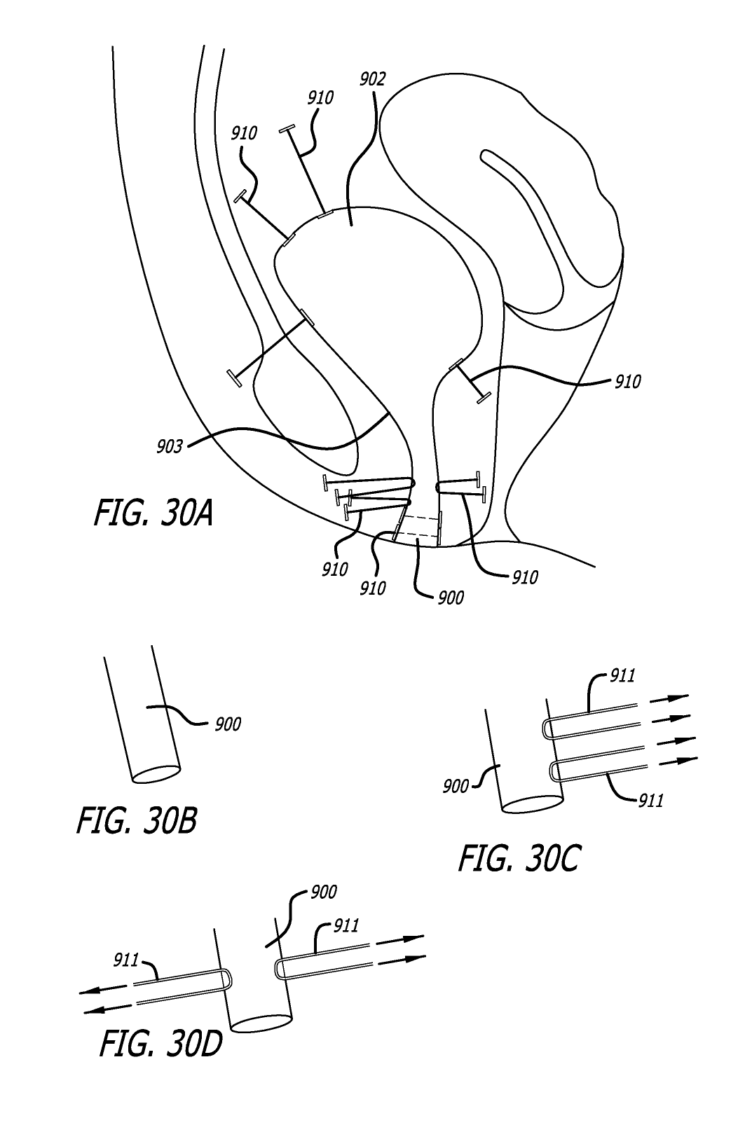

In one particular treatment method, the previously disclosed anchor device can be employed to treat female stress urinary incontinence, preferably type II. It is to be recognized, however, that the following can be employed to treat other maladies such as prolapse as well. Referring now to FIGS. 30A-30D where urethral hyper mobility often inherent in female incontinence is shown being treated. Whereas previous approaches relied upon use of the vaginal wall as a back board to close the urethra or reposition the bladder, the presently contemplated methods involve directly re-forming the urethra 900 and/or directly re-positioning the bladder 902 or bladder neck 903. The present approach also avoids involvement of the abdominal wall so that unlike before, the anchors are not knotted thereto. Accordingly, involvement of such anatomical structures is not required to treat female type II stress urinary incontinence. Thus, an urethra 900 can be reformed to define a relatively closed cross-section (See FIGS. 30C-30D) as opposed to an untreated relatively open profile (FIG. 30B) by directly attaching anchors 910 about an urethra while configuring suture 911 attached thereto to effect desired tension to close the urethra 900. The anchors alter the shape of the urethra to change a round flaccid or slack fluid lumen to an oval taught lumen.

Moreover, as best seen in FIG. 30A, the position of the bladder 902 and bladder neck 903 can be shifted to regain continence. This approach is particularly effective for stress incontinence as the bladder 902 or bladder neck 903 is repositioned with the anchor to overcome the increased intraabdominal pressure. That is, shifting the bladder/bladder neck with respect to the forces created during laughing, sneezing, or coughing addresses incontinence. Thus, as shown in FIG. 30A, the bladder 902 or bladder neck can be directly pinned with anchor assemblies 910 laterally to create a component shift in other directions (anterior, posterior, cranial, caudal). For example, anchors can be placed in or about one or more of the periostium, ureterosacral ligaments, pelvic floor, fascia, previously implanted sling, arcuate tendon and Cooper's ligament. It is also to be recognized that it can be useful to deliver an anchor beyond a target site so that sufficient space is provided for turning of the anchor and to account for tissue elasticity. Also, implantation of an anchor beyond or within a tissue plane or structure (ligament, periostium) can help to provide desired purchase.

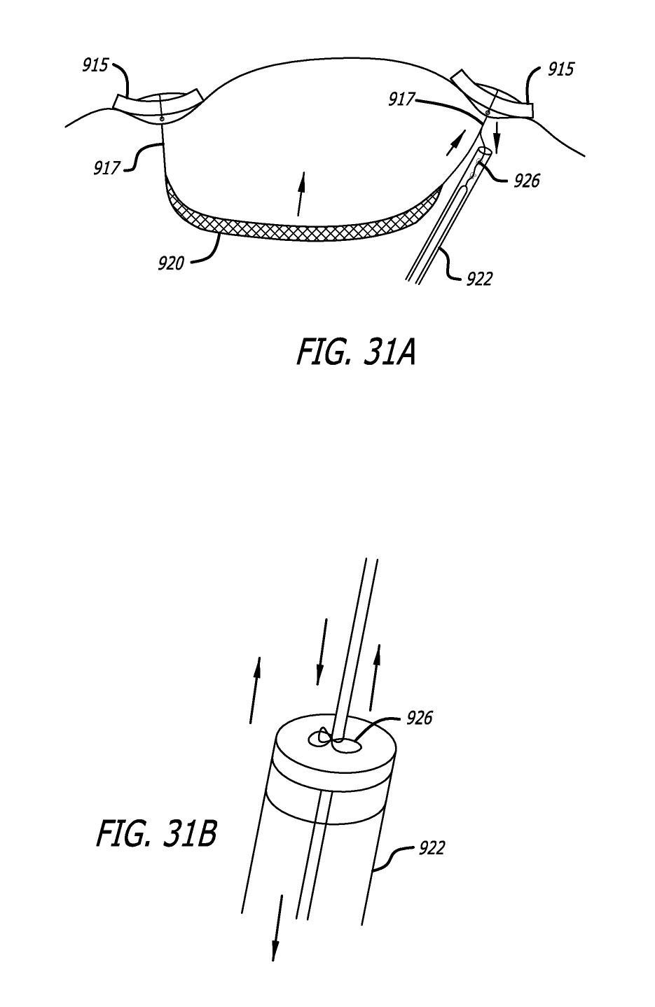

In another treatment modality (FIGS. 31A-31B), anchors 915 can be placed in tissue with a suture 917 routed between the anchors 915. The suture 917 can be placed at an external surface and can further carry a mesh structure 920. A slip knot arrangement is contemplated for this application so that applying a tension on the suture 907 causes the external suture 917 and/or mesh 920 to create a compression force in a direction generally perpendicular to implanted anchors 915. A subassembly delivery apparatus 922 can then be configured and employed to advance a lock or suture slip knot 926 against one anchor to thereby tighten the suture 917 and/or mesh 920 and retain the anchors 915, suture 917 and mesh 920 in place. Again, here, employing a slip knot or otherwise permitting the suture to move relative to the anchor provides certain versatilities. For example, various anchors can move along the suture relative to each other so that greater or lesser degrees of forces can be applied to tissue captured between the anchors.

As stated, one aspect of the present invention is the method of treating stress urinary incontinence using a tissue support such as those shown in FIGS. 1-29. This method may use a transvaginal or transperineal approach. Local anesthetic may be injected into the anterior vaginal wall in the female. The formed support structure is placed via an appropriate implantation device into the anterior vaginal wall (or pelvic floor for males), lateral to mid-urethra, or bladder neck, on both sides, in order to affect a bolstering or suspension effect of the anterior wall of the vagina (or urethra).

The tissue support structure can be implanted into the tissues that support the urethra or bladder neck. In a related embodiment, the device tightens the area around the urethra, rectum, or pelvic floor. The tissue serves to increase the support provided by the lax support structures, as such laxity contributes to incontinence.

The support structure of the present invention may be delivered and implanted in an elongated state, as illustrated in FIGS. 14A-15B, in which the implant is illustrated by member 600. After anchoring the implant in tissue by, for example, using tissue anchors as shown and described above, the device is configured to support to the urethra and bladder neck (See FIG. 30B). In addition to supporting the urethra and bladder neck by increasing the tension in the support tissues, the implant of the present invention also increases the strength of those support tissues by the integration of the implant itself into the tissues and by the tissue healing response initiated by the foreign body reaction, resulting in remodeling of the support tissues. This strengthening function can also be used to address fecal and urge incontinence as well as to simplify pelvic floor and vaginal repairs that can be performed in an office setting.

Once implanted, the anchor assembly of the disclosed embodiments accomplishes desired tissue manipulation, approximation, compression or retraction, as well as cooperates with the target anatomy to provide an atraumatic support structure. In addition to an intention to cooperate with natural tissue anatomy, the disclosed embodiments also contemplate approaches to accelerate healing or induce scarring. Manners in which healing can be promoted can include employing abrasive materials, textured connectors, biologics and drugs.

It is further contemplated that in certain embodiments, the anchor assembly can include the ability to detect forces being applied thereby or other environmental conditions. Other sensors which can detect particular environmental features can also be employed such as blood or other chemical or constituent sensors. Moreover, one or more pressure sensors or sensors providing feedback on the state of deployment of the anchor assembly during delivery or after implantation are contemplated. For example, tension, depth, relative position, or degradation feedback can be monitored by these sensors. Further, such sensors can be incorporated into the anchor assembly itself, other structure of the deployment device or in the anatomy.

The proposed structures can be connected by an element that applies supportive or expansive forces such as a metallic wire, plastic member, or dehydrated absorbable material. This element can be used to maintain distance between the two end pieces in order to provide bulking effect or scaffolding functionality. Specifically, in the application of urinary incontinence in women, the expansive device could be used to strengthen portions of the urethral wall, the vaginal wall, the rectal wall, the distance between the urethra and the pelvic floor, the distance between the bladder and the pelvic floor, etc.

The proposed elements can be placed such that there is no tension applied during delivery. This could allow for the natural structure of the tissue to be strengthened without changing the relative position of an existing tissue plane.

The proposed elements can be deployed in a manner such that the secondary anchor deploys an element not originally contained within the delivery device.

The delivery device can be designed to dilate body orifices to allow for passage of the instrument.

Finally, it is to be appreciated that the invention has been described hereabove with reference to certain examples or embodiments, but that various additions, deletions, alterations and modifications may be made to those examples and embodiments without departing from the intended spirit and scope of the disclosed embodiments. For example, any element or attribute of one embodiment or example may be incorporated into or used with another embodiment or example, unless to do so would render the embodiment or example unpatentable or unsuitable for its intended use. Also, for example, where the steps of a method are described or listed in a particular order, the order of such steps may be changed unless to do so would render the method unpatentable or unsuitable for its intended use. All reasonable additions, deletions, modifications and alterations are to be considered equivalents of the described examples and embodiments and are to be included within the scope of the following claims.

The various embodiments described above are provided by way of illustration only and should not be construed to limit the disclosed embodiments. Those skilled in the art will readily recognize various modifications and changes that may be made to the disclosed embodiments without following the example embodiments and applications illustrated and described herein, and without departing from the true spirit and scope of the disclosed embodiments, which is set forth in the following claims.

What is claimed is:

1. A system comprising:
an anchor body;
at least one resilient wing extending from the anchor body, the resilient wing is foldable against the anchor body;
a resilient fin extending from the anchor body, the resilient fin is foldable against the anchor body;
a connector attached to the anchor body; and
a delivery apparatus about which the anchor body is configured and an outer sheath sized to hold the resilient wing and the resilient fin of the anchor body in their folded configuration.

2. The system of claim 1, further comprising a delivery assembly wherein while housed in the delivery assembly and prior to deployment at a target site, the anchor body is constrained in a generally straight configuration, and wherein, upon deployment from the delivery assembly, the anchor body subsequently assumes a curved configuration.

3. The system of claim 1, wherein the anchor body is generally straight when unconstrained.

4. The system of claim 1, wherein the resilient wing includes a wire-form.

5. The system of claim 4, wherein the wire-form defines a loop.

6. The system of claim 4, wherein there are a plurality of wire-forms which define the resilient wing.

7. The system of claim 1, wherein the fin is formed from a wire-form.

8. The system of claim 1, wherein there are a plurality of wire-forms defining the fin.

9. The system of claim 1, further comprising a plurality of fins.

10. The system of claim 1, further comprising at least three wings.

11. The system of claim 1, wherein the anchor body further includes a mesh patch.

12. The system of claim 1, wherein the anchor body is bioabsorbable.

13. The system of claim 1, wherein the anchor body includes radiopaque markers.

14. The system of claim 1, wherein the delivery apparatus is configured to deploy a plurality of anchor bodies.

15. The system of claim 1, further comprising means for directly reforming a urethra or repositioning a bladder in a manner addressing female incontinence without reforming surrounding tissue.

16. The system of claim 1, further comprising means for shifting without securing a bladder and bladder neck to resist movement due to forces created during valsalva.

17. The system of claim 1, further comprising a proximal anchor component.

18. The system of claim 17, wherein the proximal anchor component defines a clothespin like structure.

19. The system of claim 18, wherein the proximal anchor component defines a tubular device with a deformable center that engages the connector.

20. The system of claim 17, wherein the proximal anchor component is defined by two pieces.

21. The system of claim 1, wherein the delivery apparatus includes a slot for engaging the connector.

22. The system of claim 1, wherein the anchor body includes a longitudinal slot adapted to receive a corresponding rail formed on the delivery apparatus.

23. The system of claim 1, wherein the anchor includes means for routing the connector along a length of the anchor.

24. The system of claim 1, wherein the anchor includes a bore configured to receive an anchor.

25. The system of claim 1, wherein the anchor includes means for turning the anchor within tissue when released from the delivery apparatus.

26. The system of claim 25, wherein the means include a turning fulcrum.

27. The system of claim 25, wherein the means is embodied in flexible gradients along the anchor body.

28. The system of claim 1, further comprising a pair of anchor bodies connected by a slip knot arrangement configured to apply a tension to a mesh in a direction perpendicular to the anchor bodies.

29. A method for introducing an anchor within a patient, comprising:
accessing a target site within an interventional site with a delivery apparatus, the delivery apparatus housing at least one anchor; and
delivering the at least one anchor beyond the target site so that sufficient space is provided for turning of the at least one anchor and to account for tissue elasticity.

30. A method for introducing an anchor within a patient, comprising:
accessing a target within an interventional site with a delivery apparatus, the delivery apparatus housing at least one anchor; and
delivering the at least one anchor beyond or within a tissue plane or structure to provide purchase.
